(19) United States
(12) Patent Application Publication
(10) Pub. No.: US 2017/0190746 A1
(43) Pub. Date: Jul. 6, 2017

(54) RECOMBINANT POLYPEPTIDE FOR PROMOTING SCARLESS WOUND HEALING AND BIOADHESIVE MATERIAL INCLUDING THE SAME

(71) Applicant: POSTECH ACADEMY-INDUSTRY FOUNDATION, Pohang-si (KR)

(72) Inventors: Hyung Joon CHA, Pohang-si (KR); Bong Hyuk CHOI, Pohang-si (KR); Eun Young JEON, Pohang-si (KR)

(21) Appl. No.: 15/394,663

(22) Filed: Dec. 29, 2016

(30) Foreign Application Priority Data
Dec. 30, 2015 (KR) 10-2015-0189634

(51) Int. Cl. C07K 14/435; A61L 24/00; A61L 24/10; C07K 7/08; A61K 8/64

(52) U.S. Cl. CPC C07K 14/43504; C07K 7/08; A61K 8/64; A61L 24/108; A61L 24/0015; A61K 38/00

(57) ABSTRACT

Provided are a recombinant polypeptide in which a small leucine-rich proteoglycan mimetic sequence is attached to a terminal of a mussel adhesive protein, a composition for wound healing including the same, a bioadhesive material, and a preparation method thereof. According to the present disclosure, the recombinant polypeptide in which the small leucine-rich proteoglycan mimetic sequence is attached to the terminal of the mussel adhesive protein has an excellent epidermal regeneration effect in which the wound site is uniformly restored by promoting rapid wound healing at the wound site when being applied to the wound site and inducing formation of collagens which are arranged and concentrated at the wound site, and thus can be usefully used as various drugs, cosmetics, and quasi-drugs.

### CROSS-REFERENCE TO RELATED APPLICATIONS

This application is based on and claims priority from Korean Patent Application No. 10-2015-0189634, filed on Dec. 30, 2015 with the Korean Intellectual Property Office, the disclosure of which is incorporated herein in its entirety by reference.

### TECHNICAL FIELD

The present disclosure relates to a recombinant polypeptide in which a small leucine-rich proteoglycan mimetic sequence is attached to a terminal of a mussel adhesive protein, a composition for wound healing including the same, a bioadhesive material, a preparation method thereof, and a wound healing or treating method.

### BACKGROUND

Collagen is a protein which is distributed throughout the body such as bone, skin, joints, and hair to be the basis of many tissues and is involved in maintaining various biological signals related with cells and a skeletal structure of the tissue. In addition, functions of the tissue may be changed according to a size, an alignment, and a structure of collagen fibril and various physical forces of various tissues (skin and bone) including collagen may be exemplified. In this regard, in order to control physical force of a collagen-based structure, many studies related to the control of collagen fibrosis according to existence of additional extracellular matrix components have been conducted. Until now, 20 types or more of collagens have been verified and type 1 is the most common type which is found in the tissues.

Decorin is small leucine-rich proteoglycan (hereinafter, SLRP) which is most commonly found in the adult skin and constituted by a protein center capable of being attached to collagen and dermatan sulfate (DS) and glycosaminoglycan (GAG) side chains. The decorin, like another SLRP, is attached to collagen through the protein center to prevent lateral aggregation occurring during a collagen fibrosis process and well-known to be involved in a skin structure and wound healing by controlling a diameter size, a fibril distribution, and an alignment of collagen fibril. Further, the decorin protects collagen from matrix metalloproteinase (MMP) which rapidly increases during wound healing to prevent the collagen from being degraded and prevent scar formation. Further, the decorin is attached to TGF β which is generated in the wound tissue to cause inflammation to reduce the amount and prevents extreme inflammation in the wound tissue. Accordingly, it is known that decorin-deficient skin has an irregular outline and a non-uniform fibril diameter to have a skin structure without functionality and unstable dermis regeneration. Further, abnormal wound healing associated with decorin deficiency leaves either hypertrophy or keloid scar. As such, functionality of the decorin associated with wound tissue healing and scar minimization is known, but since it is very difficult to isolate and extract the decorin from an animal tissue or prepare the decorin, clinical trials using the decorin are extremely limited.

A bioadhesive material means a material having adhesion to various living body parts such as cell walls, cell membranes, proteins, DNA, growth factors, and tissues and medical applications such as haemostatic agents or tissue adhesives for wound closure, tissue fillers, tissue regeneration agents, wound dressings, and drug delivery carriers are possible. However, currently, a bioadhesive material for medical care serves as an adjuvant for closing the wound generated during surgery and actually, the functionality and physical properties are lack to be used as a bioadhesive material for medical care. Most basically, since a medical adhesive is directly in contact with the tissue, biocompatibility is required and in addition to adhesion and ease in which adhesion can be instantaneously terminated in a body environment, the function needs to be maintained for a long time. As a representative bioadhesive which is currently commercialized and practical use, a cyanoacrylate-based instant adhesive, fibrin glue, a polyurethane-based adhesive, or the like is included. Cyanoacrylate is cured quickly without an initiator and has high adhesion strength, but is weak in impact, has decreased heat resistance and water resistance, and causes an immune response due to toxicity. Further, a fibrin-based bioadhesive has relatively excellent biocompatibility and biodegradability because of a method using an actual blood clotting process, but has significantly lower adhesion than a synthetic polymer-based adhesive and thus it is very limited to be used in a region requiring underwater adhesion. A polyurethane-based bioadhesive has high adhesion with the tissue and flexibility, but has a problem to reduce bio-toxicity of a synthetic raw material. As such, currently, most of adhesive materials are chemical synthesis-based materials and are weak to moisture and have toxicity and a biosynthesis-based bioadhesive material which is proposed as an alternative is largely lack in terms of adhesion.

Various crosslinking methods for preparing the bioadhesive material have been used, and most of crosslinking methods use chemical crosslinkers. As a detailed example, in the case of crosslinking of a protein using glutaraldehyde, the glutaraldehyde is known to play a role in protein crosslinking of amine groups, but known to be involved in nonspecific crosslinking of various amino acid residues such as histidine, cysteine, proline, and glycine including lysine, tyrosine, tryptophan, and phenylalanine. Most of chemical crosslinking methods including glutaraldehyde make a large change in the protein structure and have cell and tissue toxicities to be used together with cells. Accordingly, in the chemical crosslinking method, for avoiding cytotoxicity, since cells are coated or injected on an already formed support by crosslinking, a support in which the cells are uniformly distributed may be not immediately obtained. As another example, there is a photocrosslinking method, and the photocrosslinking method has been frequently used in a tissue engineering technology because it is easy to control the physical properties through light projection intensity and time and the control of the concentration of an initiator and a monomer in addition to easy accessibility and curing within short time. However, an ultraviolet (UV) polymerization method which is most commonly used is fatal to the survival of the cells and thus may not be used together with the cells. Further, these synthetic polymers formed above are less bioactive and biodegradable and less reactive with bioactive materials than protein-based materials, and furthermore, these synthetic polymers are not suitable as an in situ bioadhesive material.

Accordingly, a need for a new bioadhesive material that has no toxicity to cells and has excellent adhesion in vivo is high and researches to utilize the new bioadhesive material to promote wound healing is urgently required.

### SUMMARY

The inventors verified that a recombinant polypeptide prepared by attaching a small leucine-rich proteoglycan mimetic sequence to a mussel adhesive protein having excellent biocompatibility has excellent biocompatibility and induces rapid healing of the wound at the wound site and even recovery of the wound without scar and completed the present disclosure.

The present disclosure has been made in an effort to provide a recombinant polypeptide in which a small leucine-rich proteoglycan mimetic sequence is attached to a terminal of a mussel adhesive protein, a composition for wound healing including the same, a bioadhesive material, a preparation method thereof, and a wound healing or treating method.

An exemplary embodiment of the present disclosure provides a recombinant polypeptide in which a small leucine-rich proteoglycan mimetic sequence is attached to a terminal of a mussel adhesive protein.

Further, another exemplary embodiment of the present disclosure provides a composition including the recombinant polypeptide.

Further, yet another exemplary embodiment of the present disclosure provides a cosmetic composition for wound healing including the recombinant polypeptide.

Further, still another exemplary embodiment of the present disclosure provides a pharmaceutical composition for wound healing or treating including the recombinant polypeptide.

Further, still yet another exemplary embodiment of the present disclosure provides a quasi-drug composition for wound healing including the recombinant polypeptide.

Further, still yet another exemplary embodiment of the present disclosure provides a bioadhesive material including the recombinant polypeptide.

Further, still yet another exemplary embodiment of the present disclosure provides a preparation method of a bioadhesive material including 1) preparing a recombinant protein by attaching small leucine-rich proteoglycan to a terminal of a mussel adhesive protein.

Further, still yet another exemplary embodiment of the present disclosure provides a wound treating or healing method including administrating the recombinant polypeptide to a subject.

According to the present disclosure, the recombinant polypeptide in which the small leucine-rich proteoglycan mimetic sequence is attached to the terminal of the mussel adhesive protein has an excellent epidermal regeneration effect in which the wound site is uniformly restored by promoting rapid wound healing at the wound site when being applied to the wound site and inducing formation of collagens which are arranged and concentrated at the wound site and thus can be usefully used as various drugs, cosmetics, quasi-drugs, and biomaterials.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a diagram illustrating a result of verifying an fp-151-collagen binding peptide (fp-151-CBP) through electrophoresis.

FIG. 2 is a diagram illustrating a result of verifying a function for collagen fibrosis delay of the fp-151-CBP through turbidimetry.

FIG. 3 is a diagram illustrating a result of verifying a function for preventing collagen fibril degradation of the fp-151-CBP through an enzyme reaction.

FIG. 4 is a diagram illustrating an appearance of a solution in which a mussel adhesive protein fp-151-CBP is dissolved with 30 wt %, before photocrosslinking.

FIG. 5 is a diagram illustrating a gel form formed by irradiating a mussel adhesive protein fp-151-CBP solution including Ru(II)bpy₃²⁺ and persulfate with a dental lamp having a wavelength range of 450 nm.

FIG. 6 is a diagram illustrating a result of verifying wound protection, reduction and healing effects for a full-thickness skin tissue defect of the mouse skin by using an adhesive material formed by a photocrosslinking reaction of the fp-151-CBP.

FIG. 7 is a diagram illustrating a result of verifying a tissue regenerated at the 7-th day after generating a tissue defect through tissue staining using H&E.

FIG. 8 is a diagram illustrating a result of verifying a tissue regenerated at the 14-th day after generating a tissue defect through tissue staining using H&E.

FIG. 9 is a diagram illustrating a result of verifying a tissue regenerated at the 14-th day after generating a tissue defect through immunohistologic analysis using masson's trichrome (MT) staining.

FIG. 10 is a diagram illustrating a result of verifying a tissue regenerated at the 21-st day after generating a tissue defect through Picrosirius red staining.

FIG. 11 is a diagram illustrating a result of verifying a tissue regenerated at the 21-st day after generating a tissue defect through TEM analysis.

FIG. 12 is a diagram illustrating a result of verifying a tissue regenerated at the 28-th day after generating a tissue defect through real-time PCR analysis.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

The present disclosure provides a recombinant polypeptide in which a small leucine-rich proteoglycan mimetic sequence is attached to a terminal of a mussel adhesive protein and a composition including the same.

The recombinant polypeptide has an excellent epidermal regeneration effect in which the wound site is uniformly restored by promoting rapid wound healing at the wound site when being applied to the wound site and inducing formation of collagens which are arranged and concentrated at the wound site.

In the present disclosure, the “mussel adhesive protein” is an adhesive protein derived from mussels and preferably, includes a mussel adhesive protein derived from Mytilus edulis, Mytilus galloprovincialis or Mytilus coruscus or a variant thereof, but is not limited thereto.

For example, the mussel adhesive protein of the present disclosure may include Mytilus edulis foot protein (Mefp)-1, Mytilus galloprovincialis foot protein (Mgfp)-1, Mytilus coruscus foot protein (Mcfp)-1, Mefp-2, Mefp-3, Mgfp-3 and Mgfp-5 or variants thereof. Preferably, the mussel adhesive protein includes a protein selected from the group consisting of foot protein (fp)-1 (SEQ ID NO: 1), fp-2 (SEQ ID NO: 4), fp-3 (SEQ ID NO: 5), fp-4 (SEQ ID NO: 6), fp-5 (SEQ ID NO: 7), and fp-6 (SEQ ID NO: 8), a fusion protein in which two or more proteins are connected to each other, or a variant of the protein, but is not limited thereto.

Further, the mussel adhesive protein of the present disclosure includes all mussel adhesive proteins disclosed in International Patent Publication No. WO2006/107183 or WO2005/092920. Preferably, the mussel adhesive protein may include a fusion protein such as fp-151 (SEQ ID NO: 9), fp-131 (SEQ ID NO: 10), fp-353 (SEQ ID NO: 11), fp-153 (SEQ ID NO: 12), and fp-351 (SEQ ID NO: 13), but is not limited thereto and preferably, may be fp-151 (SEQ ID NO: 9).

Further, the mussel adhesive protein of the present disclosure may include a polypeptide in which decapeptides (SEQ ID NO: 2), which are repeated about 80 times in fp-1, are continuously connected to each other 1 to 12 times or more. Preferably, the mussel adhesive protein may be an fp-1 variant polypeptide (SEQ ID NO: 3) in which the decapeptides of SEQ ID NO: 2 are continuously repeated 12 times, but is not limited thereto.

The small leucine-rich proteoglycan (SLRP) mimetic sequence of the present disclosure includes a peptide having the same sequence as or a substantially similar sequence to a core amino acid representing SLRP functionality.

A synthetic peptide mimicking the SLRP functionality may be one kind selected from the group consisting of CQDSETRTFY (SEQ ID NO: 14), GELYKSILYGC (SEQ ID NO: 15), TKKTLRTGC (SEQ ID NO: 16), KELNLVYT (SEQ ID NO: 17), GSITTIDVPWNV (SEQ ID NO: 18), GSITTIDVPWNVGC (SEQ ID NO: 19), RLDGNEIKRGC (SEQ ID NO: 20), AHEEISTTNEGVMGC (SEQ ID NO: 21), RRANAALKAGELYKSILYGC (SEQ ID NO: 22), and the like. For example, preferably, the synthetic peptide may use RRANAALKAGELYKSILYGC (SEQ ID NO: 22), but is not limited thereto.

In the present disclosure, the synthetic peptide mimicking the SLRP functionality may be attached to an N- or C-terminal group of the existing mussel adhesive protein through PCR and the functional protein can be expressed in an Escherichia coli system like conventional mussel adhesive protein expression and purification and easily purified using acetic acid.

The recombinant polypeptide in which the small leucine-rich proteoglycan mimetic sequence is attached to the terminal of the mussel adhesive protein of the present disclosure may be a recombinant polypeptide in which one kind selected from synthetic peptides mimicking the aforementioned SLRP functionality is attached to one kind selected from the aforementioned mussel adhesive proteins. Particularly, preferably, the mussel adhesive protein may be fp-151 and the synthetic peptide mimicking the SLRP functionality may be RRANAALKAGELYKSILYGC (SEQ ID NO: 22), the recombinant polypeptide may be a recombinant polypeptide represented by SEQ ID NO: 23.

The recombinant polypeptide of the present disclosure may delay a collagen fibril fibrosis process to prevent a side aggregation process which is generated in a wound healing process to cause scars and induce preferable collagen accumulation and arrangement through collagen fibril protection and degradation prevention functions from the MMP which is collagenase increased during the wound healing process.

Particularly, the recombinant polypeptide of the present disclosure induces arrangement of collagens by comparing wound healing in a natural state and has excellence capable of inducing uniform healing of the wound without forming scars or keloids.

Accordingly, the present disclosure provides a cosmetic composition for wound healing including a recombinant polypeptide in which a small leucine-rich proteoglycan mimetic sequence is attached to a terminal of a mussel adhesive protein.

The cosmetic composition may be a formulation selected from the group consisting of emollient lotion, astringent lotion, nutrient lotion, nutrient cream, massage cream, essence, eye cream, eye essence, cleansing cream, cleansing foam, cleansing water, pack, powder, body lotion, body cream, body oil, body essence, makeup base, foundation, hair dye, shampoo, rinse and body cleanser. The cosmetic composition of the present disclosure may be prepared in various forms according to a general cosmetic preparation method using the recombinant polypeptide and may include general adjuvants such as stabilizers, solubilizers, vitamins, pigments, and perfumes which are generally used in a cosmetic composition field.

The cosmetic composition of the present disclosure may be prepared particularly in a form of skin lotion, lotion, cream, and essence, and much more preferably may be prepared in a cosmetic formulation such as scar prevention or wound healing cream. In the cosmetic composition of the present disclosure, the recombinant polypeptide of the present disclosure may be added with an amount of 0.1 wt % to 50 wt % with respect to a total liquid weight of the cosmetic composition and may be added with an amount of 0.001 to 30 wt % and preferably 0.01 to 10 wt % with respect to a total dry weight of the cosmetic composition.

Further, the functional peptide attached to the conventional mussel adhesive protein has a similar function to the SLRP and a biomaterial based on the functional peptide can be applied as medical therapeutic agents and materials for rapid and excellent tissue regeneration, scar prevention, and the like in the wound tissue.

Accordingly, the present disclosure provides a pharmaceutical composition for wound healing or treating including a recombinant polypeptide in which a small leucine-rich proteoglycan mimetic sequence is attached to a terminal of a mussel adhesive protein.

The pharmaceutical composition of the present disclosure may additionally include an additional ingredient, that is, a carrier, an excipient, a diluent or an accessory ingredient which is pharmaceutically acceptable or nutritionally acceptable according to a formulation, a use method, and a use purpose in addition to the active ingredient.

The carrier, the excipient, and the diluent may use all general things and for example, may include at least one selected from the group consisting of lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, calcium carbonate, dextrin, propylene glycol, liquid paraffin, and physiological saline, but is not limited thereto. The ingredients may be added to the active ingredient independently or in combination.

The pharmaceutical composition of the present disclosure may be administrated particularly to a wound site of a subject for external use.

Further, the present disclosure may further include an additional ingredient in order to promote wound treating and healing effects of the recombinant polypeptide of the present disclosure and an additional bioactive substance may include cells, proteins, enzymes, and the like without limitation. Particularly, as a preferable example, glycan which is a side chain component of the SLRP may be used, and the glycan configuring the SLRP may be at least one selected from the group consisting of agarose, alginate, dermatan sulfate, chondroitin, dextran, heparin, and hyaluronan.

In the case of additionally including the glycan to the recombinant polypeptide of the present disclosure, the effect of promoting the wound treating and healing of the recombinant polypeptide may be further enhanced.

Further, the present disclosure provides a quasi-drug composition for wound healing including a recombinant polypeptide in which a small leucine-rich proteoglycan mimetic sequence is attached to a terminal of a mussel adhesive protein.

The quasi-drug composition means a composition having a form such as an ointment, a patch, and a cream capable of protecting and healing the wound site.

Further, the present disclosure provides a bioadhesive material including a recombinant polypeptide in which a small leucine-rich proteoglycan mimetic sequence is attached to a terminal of a mussel adhesive protein.

The bioadhesive material of the present disclosure and an bioadhesive composition including the same are locally applied to the living body to easily and immediately adhere and close the wound by replacing a surgical suture and may be used for filling a full-thickness defect tissue caused by skin burn or surgery or regenerating the defect tissue and minimizing scars. In this specification, the term “biological tissue” is not particularly limited and for example, includes skin, nerve, brain, lung, liver, kidney, stomach, small intestine, rectum, bone, and the like.

Particularly, the bioadhesive material of the present disclosure may be a gel type. The gel type may be induced through a photocrosslinking reaction and the generated gel-type bioadhesive material is immediately applied to a full-thickness defect tissue as well as a cut and may protect an initial wound, prevent infection, and absorb an exudation generated in a regeneration process. Further, over time, a functional mussel adhesive protein emitted when the gel is gradually degraded is involved in inflammation, and production, accumulation and arrangement of collagens to be applied to a functional wound dressing for minimizing scars as well as rapid wound healing.

Accordingly, the present disclosure provides a functional wound dressing having wound healing and scar minimizing functions which is immediately applicable to a wound site including the bioadhesive material of the present disclosure and further, a drug delivery carrier is attached to a desired biological tissue without a separate adhesive to be used for inflammation prevention and rapid tissue regeneration. The drug is not particularly limited and includes protein medicines, peptides, anti-inflammatory agents, and the like.

Further, the present disclosure provides a preparation method of preparing a bioadhesive material having excellent wound treating and healing effects.

More particularly, the present disclosure provides a preparation method of a bioadhesive material including 1) preparing a recombinant protein by attaching small leucine-rich proteoglycan to a terminal of a mussel adhesive protein.

In the preparation method, the content of the mussel adhesive protein is 10 to 50 wt % and preferably 20 to 30 wt % based on the entire composition in order to induce uniform photocrosslinking, but is not limited thereto.

Further, the present disclosure provides a preparation method of a bioadhesive material including: preparing a solution dissolved with at least one material selected from the group consisting of dermatan sulfate, chondroitin, dextran, heparin, and hyaluronan; and dissolving the recombinant protein prepared in step 1) of claim 14 in the solution.

That is, when the bioadhesive material is prepared by including the glycan and the recombinant polypeptide of the present disclosure, the glycan is first dissolved so that the final concentration has the same mole number as the functional mussel adhesive protein and then the functional mussel adhesive protein is dissolved in the solvent in which the glycan is dissolved.

In the present disclosure, in order to provide the bioadhesive material including the recombinant polypeptide having the function of promoting wound healing and minimizing the scar, preferably, the bioadhesive material may be prepared by inducing a photocrosslinking reaction through light irradiation. That is, the present disclosure provides the preparation method of the bioadhesive material further including: adding a solution including photoreactive metal ligands and electron acceptors to the prepared recombinant protein and inducing a photocrosslinking reaction through light irradiation.

The photocrosslinking bioadhesive material based on the mussel adhesive protein prepared by the preparation method may be a gel type having a 3D network structure formed by crosslinking tyrosine residues included in the mussel adhesive protein.

The method is to prepare the gel-type bioadhesive material having the 3D network structure by inducing binding between tyrosine residues included in the mussel adhesive protein at a high ratio. It is known that the binding between the tyrosine residues is performed through photolysis of molecules by strongly absorbing visible light having a wavelength of 449 to 455 nm in an aqueous solution in which a metal ligand such as ruthenium tris-bipyridyldication (Ru(II)bpy₃²⁺) is dissolved. Under existence of light having a wavelength of 420 to 480 nm or 449 to 455 nm, more preferably about 452 nm, a metal complex is photolyzed in an excited state capable of giving electrons to the electron acceptor such as persulfate. As the result of the photolysis, it is known that Ru(II)bpy₃²⁺ and sulfate radicals serving as oxidants are formed. The generated Ru(II)bpy₃²⁺ forms an unstable tyrosine radical by oxidizing tyrosine in the protein aqueous solution and the radical reacts with another tyrosine residue therearound to form binding di-tyrosine. In this case, in order to form stable di-tyrosine binding, it is required to remove hydrogen atoms and it is known that the sulfate radical plays the role.

In the method, the photoreactive metal ligand for providing molecules which strongly absorb the visible light may be at least one selected from the group consisting of ruthenium (Ru (II)), palladium (Pd (II)), copper (Cu (II)), nickel (Ni (II)), manganese (Mn (II)) and iron (Fe (III)). For example, the photoreactive metal ligand may use [Ru(II)bpy₃]Cl₂, but is not limited thereto.

Further, the electron acceptor may be at least one selected from the group consisting of sodium persulfate, periodate, perbromate, perchlorate, vitamin B12, pentaamminechlorocobalt (III), ammonium cerium (IV) nitrate, oxalic acid and EDTA. For example, the electron acceptor may preferably use sodium persulfate, but is not limited thereto.

More preferably, the gel-type bioadhesive material having the 3D network structure may be formed within several seconds to several minutes when adding Ru(II)bpy²⁺ and sodium persulfate in a solution in which the mussel adhesive protein is dissolved with 10 to 50 wt % and irradiating light having a wavelength band of 420 to 480 nm.

Further, the present disclosure provides a wound treating or healing method including administrating the recombinant polypeptide to a subject.

The recombinant polypeptide is the same as those described above.

In the present disclosure, the recombinant polypeptide may be administrated by a method known in the art. The recombinant polypeptide may be directly administrated to the subject by any means as a pathway such as intravenous, intramuscular, transdermal, mucosal, intranasal, intratracheal or subcutaneous administration. The recombinant polypeptide may be administrated systemically or locally. Particularly, the recombinant polypeptide may be administrated to the wound site for external use.

In the present disclosure, the subject may be a mammal, for example, human, cow, horse, pig, dog, sheep, goat, or cat.

The “treatment” provided by the present disclosure may provide that the wound is treated for a shorter time than natural treatment. The treatment may include improving and/or alleviating the wound. Further, the treatment may include all treatments of the wound and/or diseases associated with the wound. The treatment may mean treating and/or regenerating the damaged tissue caused by the wound. The wound treatment may include a meaning of skin regeneration. Further, the treatment may maintain an original composition of the damaged tissue. Further, the treatment may promote treating and/or regenerating the damaged tissue while minimizing complications and/or scars of the diseases related with the wound.

Hereinabove, it should be interpreted that the numerical values disclosed in this specification include equivalents unless otherwise specified.

Hereinafter, the present disclosure will be described in detail by Preparation Examples and Examples. However, the following Preparation Examples and Examples just exemplify the present disclosure, and the contents of the present disclosure are not limited to the following Preparation Examples and Examples.

### Example 1. Production of Mussel Adhesive Protein Attached with Collagen-Binding Peptide

1.1 Production of Recombinant Mussel Adhesive Protein Fp-151

Fp-1 variants consisting of 6 decapeptides were synthesized so that decapeptides constituted by 10 amino acids repeated 80 times in a mussel adhesive protein fp-1 present in nature may be expressed in E. coli, and a gene (Genbank No. AAS00463 or AY521220) of Mgfp-5 was inserted between two fp-1 variants to be successfully expressed in E. coli. Thereafter, the mussel adhesive protein fp-151 was produced through a simple purification and isolation process using acetic acid (D. S. Hwang et. al., Biomaterials 28, 3560-3568, 2007). Particularly, in an amino acid sequence of fp-1 (Genbank No. Q27409 or S23760), a fp-1 variant (hereinafter, referred to as 6xAKPSYPPTYK) of SEQ ID NO: 3 in which peptides consisting of AKPSYPPTYK represented by SEQ ID NO: 2 were repeated and linked 6 times was prepared, the 6xAKPSYPPTYK was combined to an N-terminal of Mgfp-5, and the 6xAKPSYPPTYK was combined to a C-terminal of Mgfp-5 to prepare the mussel adhesive protein fp-151. The detailed preparation of the mussel adhesive protein was the same as those disclosed in International Patent Publication No. WO2006/107183 or WO2005/092920 and the prepared fp-151 was represented by SEQ ID NO: 9.

1.2 Preparation and Production of Recombinant Mussel Adhesive Protein fp-151-CBP

A sequence of RRANAALKAGELYKSILYGC (SEQ ID NO: 22) selected from a SLRP group was added to a C-terminal of the fp-151 prepared in Example 1.1 to prepare fp-151-CBP (SEQ ID NO: 23). Like fp-151, the protein was expressed by using E. coli and thereafter, acetic acid extraction and purification were performed. The purified fp-151-CBP was verified through electrophoresis and the result was illustrated in FIG. 1.

As illustrated in FIG. 1, a clear protein band was verified around 22.6 kDa as an expected molecular weight and thus it was verified that the fp-151-CBP fused with the fp-151 and the SLRP was successfully produced. The fp-151-CBP was represented by SEQ ID NO: 23.

### Example 2. Experiment for Verifying Characteristic on Collagen Fibril

2.1. Effect on Collagen Fibrosis Delay of fp-151-CBP Through Turbidity Change

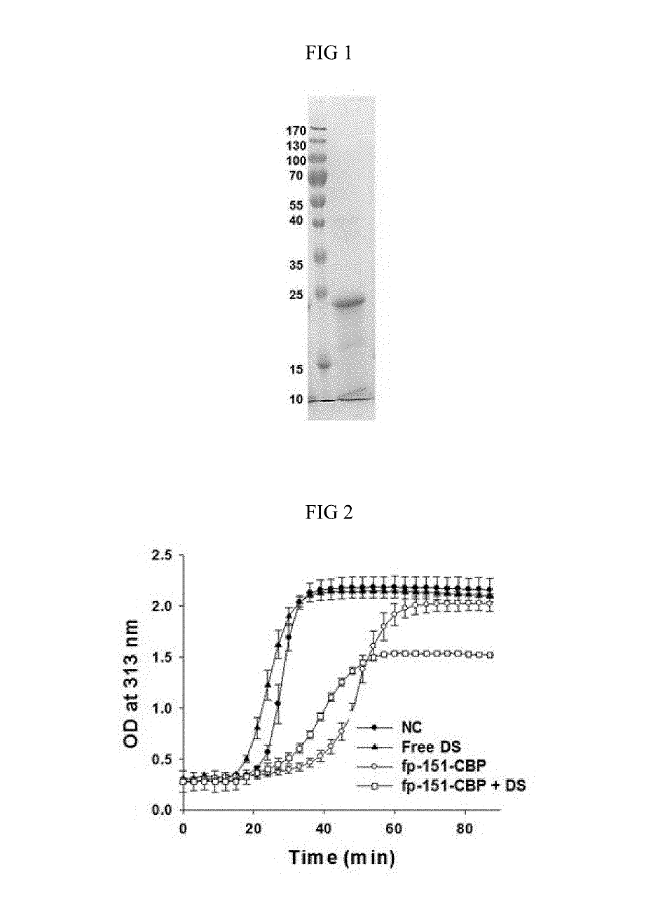

In order to verify a possibility whether the mussel adhesive protein fp-151-CBP prepared in Example 1 has an effect on collagen fibrosis delay, an experiment for fibrisis delay was performed by using Pepsin-treated type I collagen. In detail, a fp-151-CBP solution treated with fp-151-CBP dissolved in PBS and DS was treated in collagen at a final concentration of 4 mg/ml to have the same mole number as the collagen and then pH was adjusted to 7.4 by using 1M NaOH. The process was performed in ice until measuring fibrosis progression and for measuring fibrosis progression, an optical density (OD) value of a sample was measured at a wavelength of 313 nm by using UV/vis spectrometry adjusted at 37° C. As a comparative group, a collagen solution treated with only PBS was used. As a result, the collagen fibrosis delay effect was illustrated in FIG. 2.

As illustrated in FIG. 2, compared with a single-collagen treated comparative group, in an fp-151-CBP-treated group, the OD value was slowly increased and it was verified that the collagen fibrosis was delayed by the fp-151-CBP treatment.

2.2. Effect of Preventing Collagen Fibril Degradation of fp-151-CBP

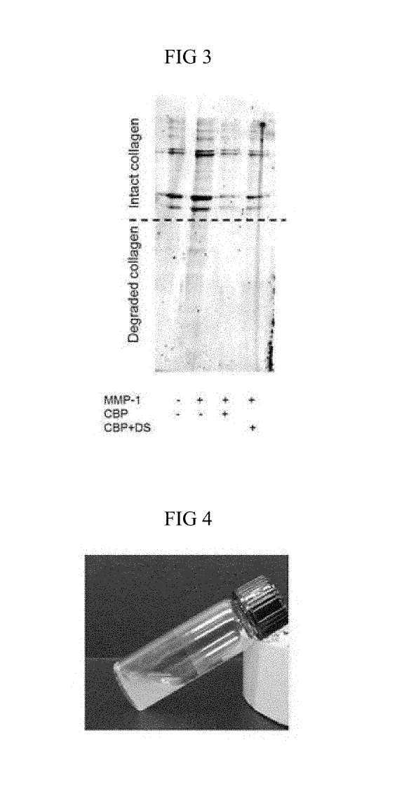

In order to verify whether fp-151-CBP has an effect of preventing collagen fibril degradation such as decorin, an experiment for preventing collagen fibril degradation was performed by using type I collagen and MMP-1 which was known that collagen was degraded during a wound healing process. In detail, for forming collagen fibril, 0.4 mg/ml of collagen dissolved in 0.1M HCl was diluted in a N-[tris(hydroxymethyl)methyl]-2-aminoethanesulfonic acid (TES) buffer at the same ratio and then stored at 37° C. for one day. In a collagen fibril pellet generated after centrifuging the generated collagen fibril solution, an fp-151-CBP solution added with the fp-151-CBP and the DS was put and then left at room temperature for 1 hr to induce collagen binding and a group treated with only distilled water was set as a comparative group. Thereafter, after centrifuging again, a degradation solution including MMP-1 was treated in the remaining pellet and stored at 32° C. for one day. In this case, as a comparative group, a degradation solution including PBS was treated. After the sample was electrophoresized by using a 7.5% polyacrylamide gel, the protein band was stained by Coomassie Orange and then verified by using UV and the result was illustrated in FIG. 3.

As illustrated in FIG. 3, in a free-fp-151-CBP sample, the collagen degraded by MMP-1 was verified, but in a sample treated with fp-151-CBP and DS-added fp-151-CBP, degraded collagen bands were not verified. As a result, it was verified that the fp-151-CBP had an effect of preventing degradation of collagen fibril.

### Example 3. Preparation of Photocrosslinking Adhesive Material

In order to prepare an adhesive material using fp-151-CBP, photocrosslinking based on visible light was induced. In detail, in order to prepare a photocrosslinking adhesive material, Ru(II)bpy₃²⁺ and a sodium persulfate solution as persulfate were added in a phosphate buffered saline (PBS) solution dissolved with the mussel adhesive protein fp-151-CBP and irradiated by a dental lamp having a wavelength band of 450 nm for 60 seconds to form a gel-type adhesive material. In more detail, Ru(II)bpy₃²⁺ at a final concentration of 1 to 2 mM and a sodium persulfate solution at a final solution of 10 to 30 Mm were added in a mussel adhesive protein aqueous solution dissolved in PBS or a 0.2 M sodium acetate buffer and then stirred well, and irradiated by a dental lamp having a wavelength band of 450 nm for 60 seconds to prepare a photoreactive gel based on the mussel adhesive protein. Further, fp-151-CBP was dissolved in PBS including dermatan sulfate (DS) of an ingredient of particularly, decorin among glycans such as agarose, alginate, dermatan sulfate, chondroitin, dextran, heparin and hyaluronan to form a gel-type adhesive material by the same method.

In detail, a photocrosslinking adhesive material was prepared at the following component concentrations:

a) dissolving 30 wt % fp-151-CBP, 1 mM Ru(II)bpy₃²⁺, and 30 mM persulfate; in PBS

b) dissolving 30 wt % fp-151-CBP, 1 mM Ru(II)bpy₃²⁺, and 30 mM persulfate; in PBS dissolved with 5 mg/ml DS

Photographs of dissolution of the mussel adhesive protein before gelation and a gel-type adhesive material after gelation were illustrated in FIGS. 4 and 5, and the gelation was visually and easily verified by adding ruthenium ions to have a yellow color. As illustrated in FIG. 5, it can be seen that the gelation is successfully performed according to the present disclosure to generate a gel-type photocrosslinking material.

### Example 4. Experiment of Wound Healing and Scar Minimization of Rat Using Photocrosslinking Material

4.1. Experiment of Wound Reduction and Healing of Rat Using Photocrosslinking Material

In order to determine how the photocrosslinking material based on the mussel adhesive protein prepared in Examples 1 to 3 had an effect on wound reduction and healing and scar generation of an actual full-thickness skin defect tissue, a wound healing experiment using a rat was performed. A photocrosslinking condition was treated to have final concentrations of fp-151-CBP 30 wt %, 1 mM Ru(II)bpy₃²⁺, and 30 mM persulfate and the solvent used PBS and PBS dissolved with 5 mg/ml DS.

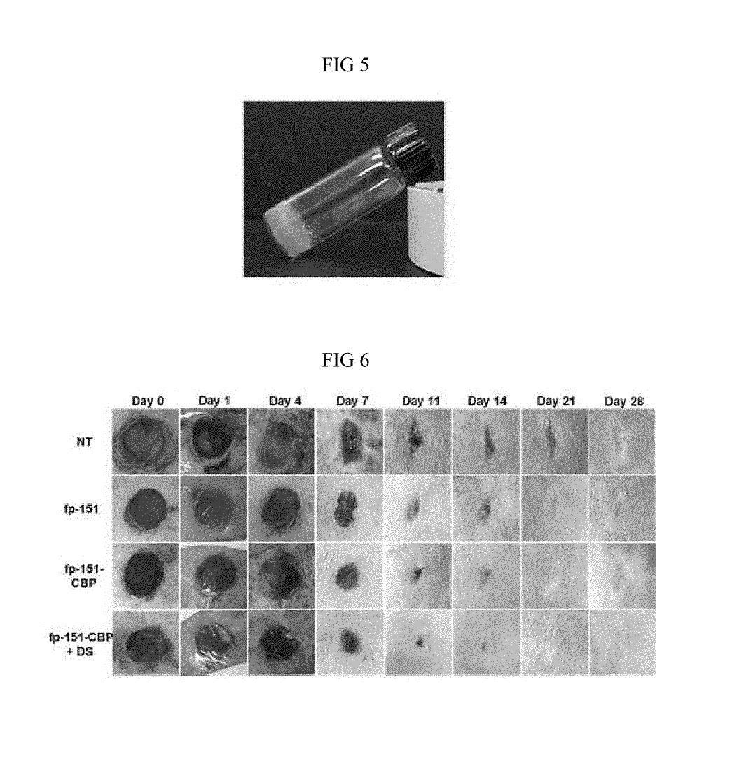

In detail, a circular full-thickness skin defect having a diameter of about 8 mm was induced on about 200 g of the back of the rat and immediately, the fp-151-CBP solution was coated on the defect site, and then a dental lamp having a wavelength band of 450 nm was irradiated for about 100 seconds to form a gel-type adhesive material. The wound healing progression was observed for 28 days and the result was illustrated in FIG. 6.

As illustrated in FIG. 6, in an experimental group treated by forming a gel of fp-151-CBP, the gel of the fp-151-CBP was attached and maintained to the wound site without a separate adhesive material and thus the fp-151-CBP-based gel may be immediately attached and maintained to the defect site. Further, as the result verified with naked eyes for about 28 days, compared with natural healing, in a defect tissue treated with materials based on the fp-151-CBP and the DS-added fp-151-CBP, rapider wound reduction and healing was verified. Further, it was verified that from the 4-th day, in fp-151-CBP and fp-151-CBP+DS-treated groups, rapider wound reduction was exhibited. In the fp-151-CBP and fp-151-CBP+DS-treated groups, the wound site was almost healed at the 14-th day, whereas in the natural healing group and GF the wound site was still verified with naked eyes.

Through the above result, it was verified that the fp-151-CBP was used as a material capable of promoting wound healing.

4.2. Experiment of Inflammation, Wound Reduction and Healing Through Dye Analysis of Healed Wound Tissue

In order to verify wound reduction and healing of a full-thickness skin defect tissue performed in 4.1, a section of the wound tissue at the 7-th day or the 14-th day after generating the defect was put in a 10% formalin solution and then tissue-stained using H&E staining, and wound reduction and healing promotion were verified according to treatment of the fp-151-CBP-based material. As a comparative group, a natural healing group was used.

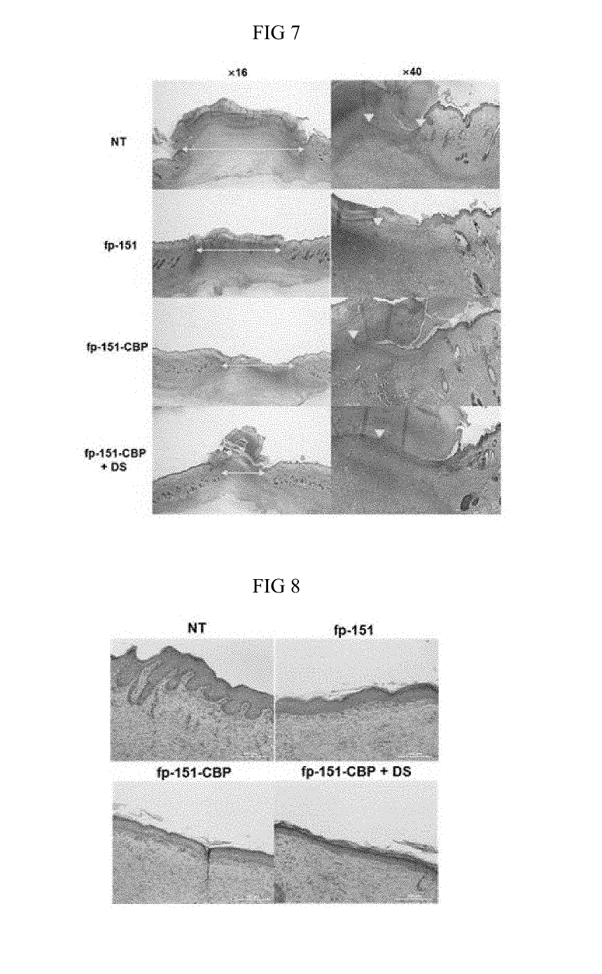

As illustrated in FIG. 7, in the natural healing group, the wound reduction was slow and uneven epidermal regeneration was exhibited, but in the group treated with the fp-151-CBP and the DS-added fp-151-CBP, an excellent epidermal regeneration effect in which the wound reduction was fast and the wound site was uniformly restored was verified. Further, in the fp-151-CBP-based material treated group, it was verified that abnormal and severe inflammation was not generated.

Further, as illustrated in FIG. 8, as compared with the natural healing group which has characteristics of the scar tissue such as non-uniform epidermal regeneration and high-level epidermal hypertrophy at the 14-th day after generating the tissue defect, in the defect tissue of the group treated with the fp-151-CBP and the DS-added fp-151-CBP-based materials, excellent epidermal regeneration and wound healing effects were verified.

Accordingly, the fp-151-CBP-based material of the present disclosure had a uniform healing effect as well as rapid wound healing in the wound healing to have a very excellent effect in wound healing.

4.3. Experiment of Verifying Collagen Generation, Accumulation, and Arrangement Through Immunohistological Analysis of Healed Wound Tissue

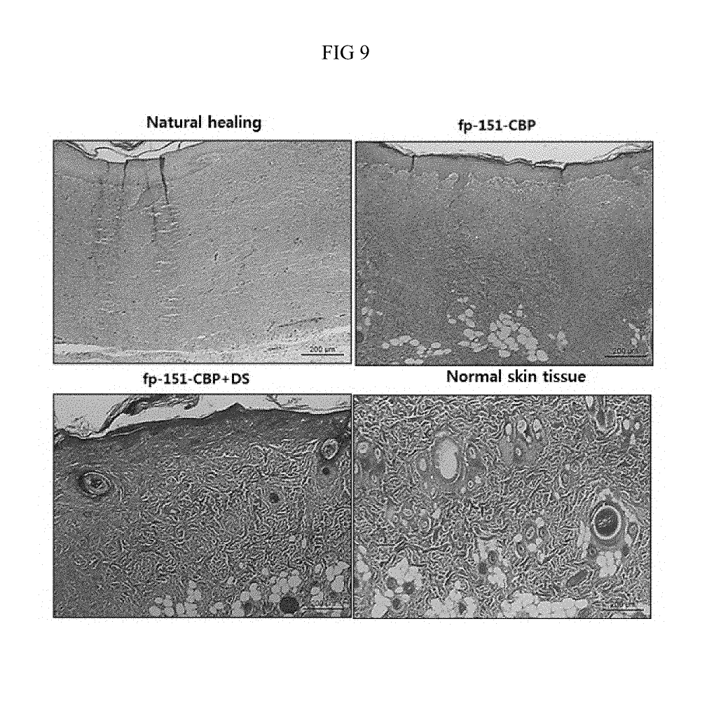

In order to verify collagen generation, accumulation, and arrangement of the full-thickness skin defect tissue performed in 4.1, a section of the wound tissue at the 14-th day after generating the defect was put in a 10% formalin solution and then immunohistological analysis using MT staining was performed and the result was illustrated in FIG. 9.

As illustrated in FIG. 9, in the natural healed wound tissue, as compared with a normal skin tissue, loose collagen generation and accumulation were verified and an unarranged appearance was verified. However, in the tissue treated with the materials based on the fp-151-CBP and the DS-added fp-151-CBP, dense collagen accumulation was verified and particularly, in the tissue treated with the material based on the DS-added fp-151-CBP, collagen arrangement similar to collagen arrangement in a normal tissue was verified.

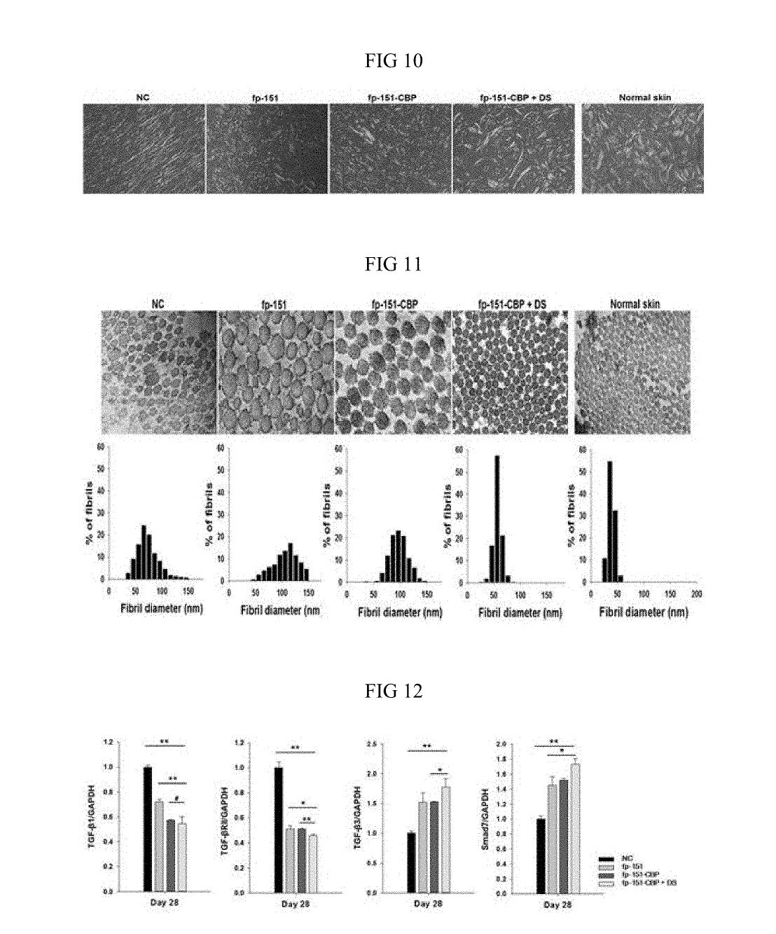

Further, the wound tissue at the 21-st day after generating the tissue defect was put in a 10% formalin solution and then stained by picrosirius red, and verified by a polarizing microscope, and the result was illustrated in FIG. 10.

As illustrated in FIG. 10, in the tissue treated with the materials based on the fp-151-CBP and the DS-added fp-151-CBP, collagen arrangement and components which were the most similar to the normal tissue were verified.

As a result, the fp-151-CBP-based material may have collagen accumulation and arrangement effects which are important factors in wound healing and scar minimization, and it is very effective that the fp-151-CBP-based material induces wound healing and recovery while suppressing traces after wound recovery such as keloid scars.

4.4. Experiment of Verifying Size, Distance, and Shape of Collagen Fiber Through TEM Analysis of Healed Wound Tissue

In order to verify a size, a distance, and a shape of collagen fiber of the full-thickness skin defect tissue performed in 4.1, a section of the wound tissue at the 21-st day after generating the defect was put in a 10% formalin solution and then TEM analysis was performed, and the result was illustrated in FIG. 11.

As illustrated in FIG. 11, in the natural healing group, abnormal focal fusion and the like as well as non-uniform size, distance, and shape of collagen fiber in the wound tissue were verified, whereas in the tissue treated with the materials based on the fp-151-CBP and the DS-added fp-151-CBP, collagen fiber having uniform size, distance, and shape was observed and it was verified that the most similar appearance to collagen fiber of the normal tissue was exhibited.

The result exhibits that the fp-151-CBP-based material has an effect of restoring the size, distance, and shape of collagen fiber which are important factors in wound recovery and scar minimization.

4.5. Experiment of Verifying mRNA Expression of Profibrotic Factor and Anti-Fibrotic Factor Through RT-PCR Analysis of Healed Wound Tissue

In order to verify mRNA expression of a profibrotic factor and an anti-fibrotic factor in the full-thickness skin defect tissue performed in 4.1, a section of the wound tissue at the 28-th day after generating the defect was put in a Trizol solution and then RT-PCR analysis was performed after cDNA synthesis. The result was illustrated in FIG. 12.

As illustrated in FIG. 12, in the tissue treated with the materials based on the fp-151-CBP and the DS-added fp-151-CBP, as compared with the natural healed tissue, it was verified that mRNA expression of TGF-β1 and TGF-β receptor II as the profibrotic factor was inhibited and mRNA expression of TGF-β3 and Smad7 as the anti-fibrotic factor was improved.

Accordingly, it is verified that the fp-151-CBP-based material has wound recovery and scar minimization effects by inhibiting the expression of the profibrotic factor and improving the expression of the anti-fibrotic factor in the skin defect tissue.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A recombinant polypeptide wherein a small leucine-rich proteoglycan mimetic sequence is attached to a terminal of a mussel adhesive protein.

2. The recombinant polypeptide of claim 1, wherein the mussel adhesive protein is at least one selected from the group consisting of fp-1 (SEQ ID NO: 1), fp-2 (SEQ ID NO: 4), fp-3 (SEQ ID NO: 5), fp-4 (SEQ ID NO: 6), fp-5 (SEQ ID NO: 7), fp-6 (SEQ ID NO: 8), fp-151 (SEQ ID NO: 9), fp-131 (SEQ ID NO: 10), fp-353 (SEQ ID NO: 11), fp-153 (SEQ ID NO: 12), and fp-351 (SEQ ID NO: 13).

3. The recombinant polypeptide of claim 1, wherein the small leucine-rich proteoglycan is at least one selected from the group consisting of polypeptides represented by SEQ ID NOs: 14 to 22.

4. The recombinant polypeptide of claim 1, wherein the mussel adhesive protein is fp-151 (SEQ ID NO: 9) and the small leucine-rich proteoglycan mimetic sequence is a polypeptide represented by SEQ ID NO: 22.

5. The recombinant polypeptide of claim 4, wherein the recombinant polypeptide is a polypeptide represented by SEQ ID NO: 23.

6. A composition including the recombinant polypeptide of claim 1.

7. The composition of claim 6, further comprising:
at least one material selected from the group consisting of agarose, alginate, dermatan sulfate, chondroitin, dextran, heparin, and hyaluronan.

8. The composition of claim 6, wherein the recombinant polypeptide induces uniform healing of a wound.

9. The composition of claim 6, wherein the composition is a cosmetic composition for wound healing.

10. The composition of claim 6, wherein the composition is a pharmaceutical composition for wound healing or treating.

11. A quasi-drug composition for wound healing including the recombinant polypeptide of claim 1.

12. A bioadhesive material including the recombinant polypeptide of claim 1.

13. The bioadhesive material of claim 12, wherein the bioadhesive material is a gel type.

14. A preparation method of a bioadhesive material, comprising: 1) preparing a recombinant protein by attaching small leucine-rich proteoglycan to a terminal of a mussel adhesive protein.

15. The preparation method of claim 14, further comprising:
preparing a solution dissolved with at least one material selected from the group consisting of agarose, alginate, dermatan sulfate, chondroitin, dextran, heparin, and hyaluronan; and dissolving the recombinant protein prepared in step 1) of claim 14 in the solution.

16. The preparation method of claim 14, further comprising: 2) adding a solution including photoreactive metal ligands and electron acceptors to the recombinant protein prepared in step 1) and inducing a photocrosslinking reaction through light irradiation.

17. The preparation method of claim 16, wherein the metal ligand is at least one selected from the group consisting of ruthenium (Ru (II)), palladium (Pd (II)), copper (Cu (II)), nickel (Ni (II)), manganese (Mn (II)) and iron (Fe (III)).

18. The preparation method of claim 16, wherein the electron acceptor is at least one selected from the group consisting of sodium persulfate, periodate, perbromate, perchlorate, vitamin B12, pentaamminechlorocobalt (III), ammonium cerium (IV) nitrate, oxalic acid and EDTA.

19. A wound healing or treating method, comprising: administrating the recombinant polypeptide of claim 1 to a subject.


SEQUENCE LISTING (23 sequences)

SEQ ID NO: 1 - protein, 800 aa, Artificial Sequence
  fp-1
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYK

SEQ ID NO: 2 - protein, 10 aa, Artificial Sequence
  fp-1 variant fragment
AKPSYPPTYK

SEQ ID NO: 3 - protein, 120 aa, Artificial Sequence
  fp-1 variant
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYK

SEQ ID NO: 4 - protein, 472 aa, Artificial Sequence
  fp-2
LFSFFLLLTCTQLCLGTNRPDYNDDEEDDYKPPVYKPSPSKYRPVNPCLKKPCKYNGVCKPRGGSYKCFCKGGYYGYNCNLKNACKPNQCKNKSRCVPVGKTFKCVCRNGNFGRLCEKNVCSPNPCKNNGKCSPLGKTGYKCTCSGGYTGPRCEVHACKPNPCKNKGRCFPDGKTGYKCRCVDGYSGPTCQENACKPNPCSNGGTCSADKFGDYSCECRPGYFGPECERYVCAPNPCKNGGICSSDGSGGYRCRCKGGYSGPTCKVNVCKPTPCKNSGRCVNKGSSYNCICKGGYSGPTCGENVCKPNPCQNRGRCYPDNSDDGFKCRCVGGYKGPTCEDKPNPCNTKPCKNGGKCNYNGKIYTCKCAYGWRGRHCTDKAYKPNPCVVSKPCKNRGKCIWNGKAYRCKCAYGYGGRHCTKKSYKKNPCASRPCKNRGKCTDKGNGYVCKCARGYSGRYCSLKSPPSYDDDEY

SEQ ID NO: 5 - protein, 50 aa, Artificial Sequence
  fp-3
PWADYYGPKYGPPRRYGGGNYNRYGRRYGGYKGWNNGWKRGRWGRKYYGS

SEQ ID NO: 6 - protein, 750 aa, Artificial Sequence
  fp-4
YGRRYGEPSGYANIGHRRYYERAISFHRHSHVHGHHLLHRHVHRHSVLHGHVHMHRVSHRIMHRHRVLHGHVHRHRVLHNHVHRHSVLHGHVHRHRVLHRHVHRHNVLHGHVHRHRVLHKHVHNHRVLHKHLHKHQVLHGHVHRHQVLHKHVHNHRVLHKHLHKHQVLHGHVHTHRVLHKHVHKHRVLHKHLHKHQVLHGHIHTHRVLHKHLHKHQVLHGHVHTHRVLHKHVHKHRVLHKHLHKHQVLHGHVHMHRVLHKHVHKHRVLHKHVHKHHVVHKHVHSHRVLHKHVHKHRVEHQHVHKHHVLHRHVHSHHVVHSHVHKHRVVHSHVHKHNVVHSHVHRHQILHRHVHRHQVVHRHVHRHLIAHRHIHSHQAAVHRHVHTHFEGNFNDDGTDVNLRIRHGIIYFGGNTYRLSGGRRRFMTLWQECLESYGDSDECFVQLLEGNQHLFTVVQGHHSTSFRSDLSNDLHPDNNIEQIANDHVNDIAQSTDGDINDFADTHYNDVAPIADVHVDNIAQTADNHVKNIAQTAHHHVNDVAQIADDHVNDIGQTAYDHVNNIGQTADDHVNDIAQTADDHVNAIAQTADDHVNAIAQTADDHVNDIGDTANSHIVRVQGVAKNHLYGINKAIGKHIQHLKDVSNRHIEKLNNHATKNLLQSALQHKQQTIEREIQHKRHLSEKEDINLQHENAMKSKVSYDGPVFNEKVSVVSNQGSYNEKVPVLSNGGGYNGKVSALSDQGSYNEGYAY

SEQ ID NO: 7 - protein, 82 aa, Artificial Sequence
  fp-5
KHHHHHHSSEEYKGGYYPGNTYHYHSGGSYHGSGYHGGYKGKYYGKAKKYYYKYKNSGKYKYLKKARKYHRKGYKKYYGGSS

SEQ ID NO: 8 - protein, 103 aa, Artificial Sequence
  fp-6
IAALCGIVKSIDSDDSDYDYKGRGYCTNKGCRSGYNYFGNKGYCKYGEKSYTYNCNSYAGCCLPRNPYGKLKYYCTNKYGCPNNYYFYNNKGYYYLEHHHHHH

SEQ ID NO: 9 - protein, 200 aa, Artificial Sequence
  fp-151
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGSSGSAKPSYPPTYKAKPSYPPTYKAKPSYPPTYKAKPSYPPTYKAKPSYPPTYKAKPSYPPTYKL

SEQ ID NO: 10 - protein, 171 aa, Artificial Sequence
  fp-131
AKPSYPPTYKAKPSYPPTYKAKPSYPPTYKAKPSYPPTYKAKPSYPPTYKAKPSYPPTYKPWADYYGPKYGPPRRYGGGNYNRYGRRYGGYKGWNNGWKRGRWGRKYYGSAKPSYPPTYKAKPSYPPTYKAKPSYPPTYKAKPSYPPTYKAKPSYPPTYKAKPSYPPTYKL

SEQ ID NO: 11 - protein, 175 aa, Artificial Sequence
  fp-353
PWADYYGPKYGPPRRYGGGNYNRYGRRYGGYKGWNNGWKRGRWGRKYYPWSSEEYKGGYYPGNTYHYHSGGSYHGSGYHGGYKGKYYGKAKKYYYKYKNSGKYKYLKKARKYHRKGYKKYYGGSSGSADYYGPKYGPPRRYGGGNYNRYGRRYGGYKGWNNGWKRGRWGRKYYGS

SEQ ID NO: 12 - protein, 187 aa, Artificial Sequence
  fp-153
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGSSGSADYYGPKYGPPRRYGGGNYNRYGRRYGGYKGWNNGWKRGRWGRKYYGS

SEQ ID NO: 13 - protein, 187 aa, Artificial Sequence
  fp-351
PWADYYGPKYGPPRRYGGGNYNRYGRRYGGYKGWNNGWKRGRWGRKYYPWSSEEYKGGYYPGNTYHYHSGGSYHGSGYHGGYKGKYYGKAKKYYYKYKNSGKYKYLKKARKYHRKGYKKYYGGSSGSAKPSYPPTYKAKPSYPPTYKAKPSYPPTYKAKPSYPPTYKAKPSYPPTYKAKPSYPPTYK

SEQ ID NO: 14 - protein, 10 aa, Artificial Sequence
  small leucine-rich proteoglycan mimetic sequence
CQDSETRTFY

SEQ ID NO: 15 - protein, 11 aa, Artificial Sequence
  small leucine-rich proteoglycan mimetic sequence
GELYKSILYGC

SEQ ID NO: 16 - protein, 9 aa, Artificial Sequence
  small leucine-rich proteoglycan mimetic sequence
TKKTLRTGC

SEQ ID NO: 17 - protein, 8 aa, Artificial Sequence
  small leucine-rich proteoglycan mimetic sequence
KELNLVYT

SEQ ID NO: 18 - protein, 12 aa, Artificial Sequence
  small leucine-rich proteoglycan mimetic sequence
GSITTIDVPWNV

SEQ ID NO: 19 - protein, 14 aa, Artificial Sequence
  small leucine-rich proteoglycan mimetic sequence
GSITTIDVPWNVGC

SEQ ID NO: 20 - protein, 11 aa, Artificial Sequence
  small leucine-rich proteoglycan mimetic sequence
RLDGNEIKRGC

SEQ ID NO: 21 - protein, 15 aa, Artificial Sequence
  small leucine-rich proteoglycan mimetic sequence
AHEEISTTNEGVMGC

SEQ ID NO: 22 - protein, 20 aa, Artificial Sequence
  small leucine-rich proteoglycan mimetic sequence
RRANAALKAGELYKSILYGC

SEQ ID NO: 23 - protein, 220 aa, Artificial Sequence
  fp-151-CBD
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGSSGSAKPSYPPTYKAKPSYPPTYKAKPSYPPTYKAKPSYPPTYKAKPSYPPTYKAKPSYPPTYKLRRANAALKAGELYKSILYGC